(19) United States
(12) Patent Application Publication
(10) Pub. No.: US 2009/0082739 A1
(43) Pub. Date: Mar. 26, 2009

(54) ADJUSTABLE EYE DROP METERING DEVICE

(76) Inventor: Jonathan H. Cress, Santa Cruz, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/859,605

(22) Filed: Sep. 21, 2007

(51) Int. Cl. A61M 35/00

(52) U.S. Cl. 604/298

(57) ABSTRACT

A drop metering device for use in combination with an eye dropper or other liquid dispenser comprises a rigid receptacle having an open chamber for removably receiving a squeeze bottle of the dropper. The plunger is mounted to reciprocate radially inwardly with respect to the rigid receptacle so that, when the squeeze bottle is in place, a controlled deflection can be induced in the squeeze bottle. The plunger may be mounted on a lever arm with a ring adjustment mechanism which provides a variable stop surface between the lever arm and the receptacle. Alternatively, the plunger may be mounted in a radially oriented slide bearing with a threadably mounted stop member for adjusting the length of travel.

### BACKGROUND OF THE INVENTION

The present invention relates generally to medical apparatus and methods. More particularly, the present invention relates to devices and methods for controlling drop dispensing from squeeze bottle dispensers, particularly for delivery of eye drops to the eye.

As with any medical procedure involving delivery of drugs or other agents to an organ, dispensing eye drops to an eye benefits greatly from controlling the volume of liquid dispensed into the eye at each application. Eye drops are most commonly delivered using squeeze bottle dispensers where a patient leans his or her head back, locates the dispenser over the eye, and squeezes the bottle to dispense one or more drops into the eye. It will be appreciated that such dispensing of drops can suffer from significant inconsistency in both the volume and number of drops dispensed with any given “squeeze.”

For these reasons, it would be desirable to provide devices and methods for helping the patient to control the volume and number of drops dispensed from squeeze bottle dispensers. In particular, it would be desirable if the device and method limited the production of any single squeeze to a single drop or selected number of drops, as well as controlling volume of each drop. It would be further desirable if such devices and methods allowed the patient or physician to control the volume and to adjust for differences in viscosity, density, and other factors which might effect the volume delivered. At least some of these objectives will be met by the inventions described herein below.

### BRIEF SUMMARY OF THE INVENTION

The present invention provides both devices and methods for the improved dispensation of drops from squeezable bottle drop dispensers. Such drop dispensers will include a squeezable bottle having a dispensing tip at one end, where the bottle is filled with a liquid desired to be delivered to the eye or elsewhere and where the patient or other user can dispense a drop by inverting the bottle so that dispenser tip is pointing downward and then squeezing the bottle to cause a drop to be dispensed from the tip. The construction of such squeezable bottle drop dispensers is well-known in the pharmaceutical arts and well-described in the medical and patent literature. Exemplary constructions are included in U.S. Pat. Nos. 4,111,200; 4,553,686; and 5,558,653, the full disclosures of which are incorporated herein by reference.

The present invention is particularly directed at a drop metering device which can be used with many if not all conventional squeeze bottle drop dispensers. The drop metering device comprises a rigid receptacle having an open chamber for removably receiving the bottle of the squeeze bottle dispenser along a central axis. A plunger is mounted on the receptacle to reciprocate over a controlled length in a lateral direction relative to the central axis. In this way, an inward reciprocation of the plunger will deflect a side wall of the bottle by the controlled length to produce one or more drops having a controlled volume. As the length of travel of the plunger is controlled (although it is optionally adjustable as described below), the amount of deflection of the squeeze bottle and therefore the volume of the resulting drop(s) will be highly consistent each time the plunger is reciprocated. Advantageously, the drop metering device is reusable with most if not all squeeze bottle dispensers having similar dimensions. In some instances, however, it may be useful to incorporate the metering device as an integral part of the squeeze bottle dispenser.

In the exemplary embodiments, the receptacle is cylindrical and the chamber has a diameter selected to receive the squeeze bottle with an interference fit. It will be appreciated, however, that other means for holding or latching the bottle within the receptacle could also be provided, including straps, detents, tapes, latches, bayonet fittings, threaded fittings, and the like. Desirably, the receptacle will include a feature which limits the depth to which the bottle can be inserted into the chamber, typically being a bottom formed at one end of the cylindrical receptacle.

In a first illustrated embodiment of the present invention, the plunger comprises a lever arm having a remote end which is pivotally attached to the receptacle. The plunger is formed on a proximate end of the lever arm which is spaced apart from the remote pivoted it end that the plunger will travel in and out, along a generally lateral direction, in order to engage and deflect a wall of the squeeze bottle held in the receptacle, as the lever arm is depressed. An adjustment mechanism will usually be provided for controlling the length of travel of the plunger in the lateral direction. In the exemplary embodiment, the adjustment mechanism comprises a rotatable ring disposed between the receptacle and the lever arm. The ring will have a variable thickness so that rotation of the ring provides a variable limit on the length of travel of the plunger. Usually, the ring will have a continuously varying thickness from a maximum which prevents the plunger from deflecting into the bottle and a minimum which allows a maximum deflection of the bottle. In an alternate embodiment, the ring may include a plurality of posts having different lengths where individual posts allow the lever to deflect by different amounts.

In a second embodiment of the device of the present invention, the plunger is slidably received in a laterally oriented slide bearing disposed on the side of the receptacle. Typically, a button is provided at one end of the plunger so that the button may be depressed to push the plunger laterally inwardly into the bottle held in the receptacle. Usually, an adjustment mechanism for controlling the length of travel of the plunger in the side bearing will be provided. In the exemplary embodiment, the adjustment mechanism comprises a stop which is threadably received on a shaft of the plunger. In this way, the stop can be rotated to axially travel along the length of the shaft to vary the available length which remains for the plunger to be depressed toward the bottle.

The present invention also provides methods for dispensing a drop from a dropper. The methods comprise inserting the bottle of the dropper into a rigid receptacle. A plunger on the rigid receptacle may then be depressed by a controlled length in a laterally inward direction relative to the receptacle and toward the bottle. The plunger will thus deflect a side wall of the bottle by the controlled length to produce a single drop having a controlled volume. Usually, the method will further comprise adjusting the controlled length of travel of the plunger. In this way, the controlled length may be adjusted in the range from 0.5 mm to 7.5 mm, preferably from 1 mm to 5 mm, to produce one or more drops having a volume in the range from 10 μl to 60 μl, preferably from 20 μl to 40 μl.

### BRIEF DESCRIPTION OF THE DRAWINGS

FIG. 1 is a perspective view of a drop metering device constructed in accordance with the principles of the present invention shown adjacent to a squeeze bottle drop dispenser.

FIG. 2 is a cross-sectional view illustrating the assembly of the drop metering device and squeeze bottle of FIG. 1.

FIGS. 3A-3C illustrate how an adjustment mechanism of the drop metering device of FIGS. 1 and 2 may be used to control the side wall deflection of the squeeze bottle and therefore, the speed and volume of the drop which is dispensed.

FIG. 4 illustrates an alternative adjustment mechanism which could be used with the drop metering device of FIGS. 1 and 2.

FIG. 5 illustrates an alternative drop metering device employing a plunger which is mounted in a laterally oriented slide bearing, shown in cross-section.

FIG. 6 is a perspective view of the drop metering device of FIG. 5.

FIGS. 7A and 7B are detailed views illustrating how the adjustment mechanism of the drop metering device of FIGS. 5 and 6 controls the length of travel of the plunger.

### DETAILED DESCRIPTION OF THE INVENTION

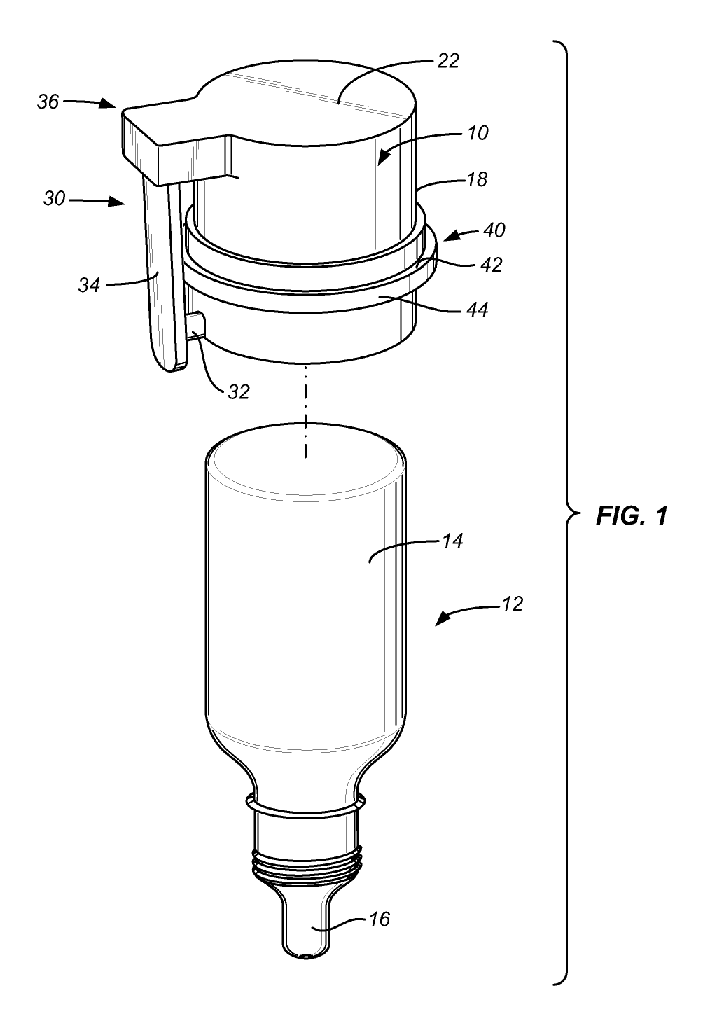

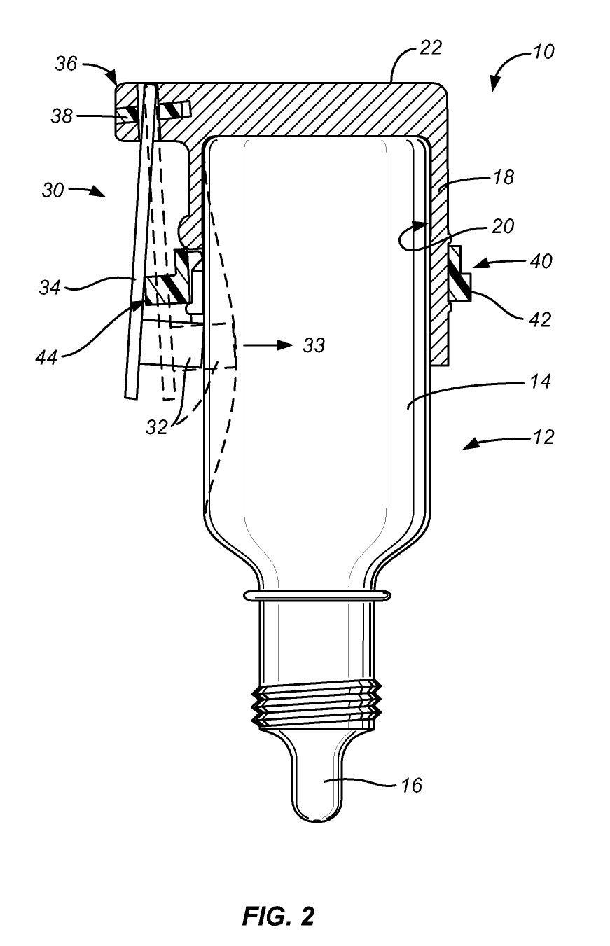

Referring to FIGS. 1 and 2, the present invention provides a drop metering device 10 for use in combination with a dropper, such as a conventional eye dropper, comprising a squeeze bottle 14 and a dispensing tip 16. The drop metering device 10 includes a receptacle 18 having an open chamber 20 for removably receiving the squeeze bottle 14 of the dropper 12, as best seen in FIG. 2. Typically, the squeeze bottle will be held in the place by friction, i.e., it will have a “friction fit.” Optionally, the use of detents, straps, tapes, bayonet fittings, threaded fittings, latches, or the like, could be used to hold the bottle in place within the receptacle 18. The receptacle 18 will be generally rigid relative to the squeeze bottle. That is, the receptacle 18 will have a significantly greater hoop strength which will prevent it being squeezed or crushed as the metering device is used by the user. As will be described in more detail below, it is necessary that the receptacle 18 provide a relatively dimensionally stable platform for lateral deployment of plunger 32 in order to provide the repeatable volume delivery which is an objective of the present invention. Receptacle 18 will also include some feature or mechanism for controlling the depth of insertion of the squeeze bottle 14 into the open chamber 20. Typically, the feature will be closed or partially closed bottom 22 so that the user may insert the squeeze bottle until it engages an interior wall of bottom.

The present invention will provide a dispensing mechanism 30 for delivering a controlled and repeatable number and/or volume of drops. Usually, the dispensing mechanism 30 will include a plunger 32 which is configured and suspended to laterally reciprocate relative to a central axis of the receptacle 18 and of the squeeze bottle 14. Viewed another way, the lateral reciprocation of the plunger 32 will be generally normal or perpendicular to the wall of the squeeze bottle, as shown by arrow 33 in FIG. 2.

In a first embodiment of the dispensing mechanism of the present invention, a lever arm 34 is suspended from a pivot mechanism 36 located at a remote or opposite end of the lever arm. Conveniently, the pivot mechanism 36 may be secured in the bottom 22 of the receptacle 18. As illustrated, the pivot mechanism 36 comprises a simple elastomeric or other spring element 38 which allows the lever arm to pivot back and forth between a disengaged configuration, as shown in full line and an engaged configuration as shown in broken line, in FIG. 2. By depressing the lever arm 34 and engaging the plunger 32 against the side wall of the squeeze bottle 14, the side wall will become deflected, as shown in broken line in FIG. 2, thus causing a volume of the liquid to be dispensed. As the length of travel of the plunger 32 is carefully controlled, as described below, the volume and/or number of drops of the liquid being dispensed will be repeatably controlled.

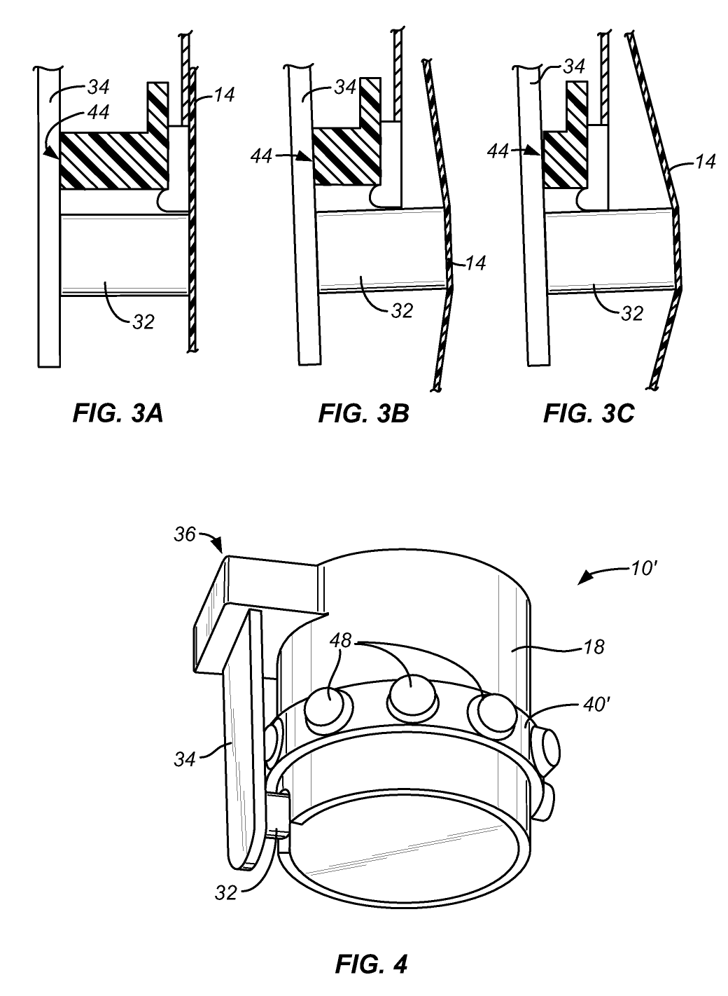

In the embodiment of FIGS. 1 and 2, an adjustment mechanism 40 is provided in order to control the length of travel of the plunger 32. The adjustment mechanism 40 includes a rotatable ring 42 having an engaging surface 44, as best seen in FIGS. 2 and 3A through 3C. A thickness of the rotatable ring 42 varies so that as the ring is rotated circumferentially over the outer wall of the receptacle 18, the engaging surface 44 will be at a variable distance from the wall of the receptacle 18. As shown in FIG. 3A, the ring 42 is rotated so that a maximum thickness is present between the lever arm 34 and the wall of the squeeze bottle 14. In this configuration, the lever arm 34 cannot be advanced radially inwardly, the bottle cannot be squeezed, and therefore no drop may be dispensed. By rotating ring 42, a portion of the ring having a lesser thickness can be interposed between the lever arm 34 and the wall of the squeeze bottle 14, as best seen in FIG. 3B. In such a configuration, the lever arm can be squeezed to cause the plunger 32 to engage and deflect the wall of the squeeze bottle 14. By further rotating the ring 42, a minimum thickness of the ring may be presented, as shown in FIG. 3C. In this position, the ring 42 allows a maximum inward travel of the lever arm 34, thus allowing the plunger 32 to deflect the side wall by a maximum distance. Thus, a maximum drop volume and speed may be produced by the metering device 10. For common eye drop dispensers having volumes in the range from 1 ml to 30 ml, a maximum plunger distance in the range from 0.5 mm to 7.5 mm will typically be provided. This allows the dispenser to deliver droplets in the range from 20 μl to 60 μl.

Referring now to FIG. 4, an alternative construction of a rotatable ring 42′ is illustrated. The rotatable ring 42′ is rotatably mounted on the outer surface of the receptacle 18, as with the prior arm embodiment. Instead of a variable thickness, the ring 42′ has a plurality of posts 48, where at least some of the posts have different heights. By rotating the ring 42′ so that different posts are interposed between the lever arm 34 and the receptacle 18, the length of travel of the plunger 32 may be controlled. This allows the users to control volume delivery in a step-wise rather than continuous fashion.

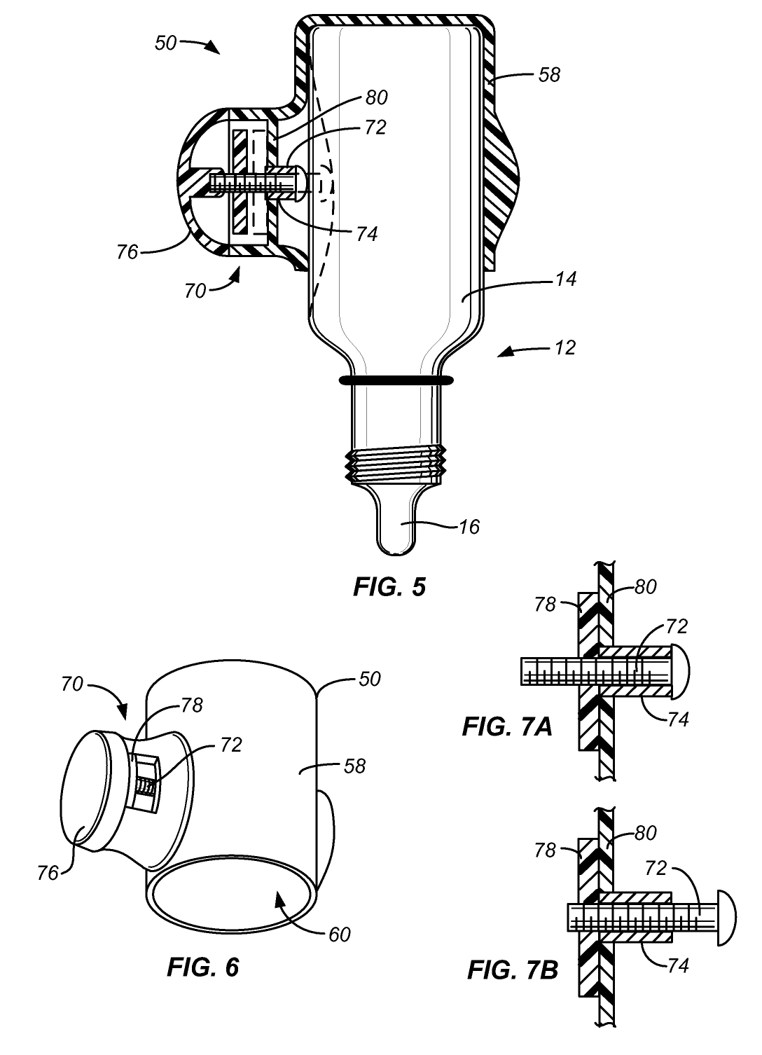

Referring now to FIGS. 5 and 6, an alternative drop metering device 50 for mounting on a dropper 12 including a squeeze bottle 14 and a dispenser tip 16 will be described. The drop metering device 50 includes a receptacle 58 having an open chamber 60 for removably receiving the squeeze bottle 14 of the dropper, generally as described with respect to the first embodiment. Drop metering device 50, in contrast, has a dispensing mechanism 70 which differs in construction although not in ultimate function. The dispensing mechanism 70, includes a plunger 72 mounted to reciprocate in a slide bearing 74 between a retracted configuration (shown in full line) and an extended configuration (shown in broken line) is seen in FIG. 5. The plunger 72 may be actuated using an elastomeric button assembly 76 which may be manually depressed. The length or depth of travel of the plunger 72 may be controlled using a stop member 78 which is threadably mounted on a shaft of plunger 72, as best seen in FIGS. 7A and 7B. The stop member 78 is usually in the form of a disk which may be manually rotated over the shaft of plunger 72, allowing the stop member to be axially translated relative to the shaft. Thus, in a distally advanced configuration, as shown in FIG. 7A, the stop member 78 is advanced fully to the right and engages a fixed wall 80 of the dispensing mechanism 70. In this position, the plunger 72 may not may not be advanced. By rotating the stop member 78 so that it travels in a proximal or radially outward direction over the shaft of plunger 72, as shown in FIG. 7B, the plunger may be radially inwardly advanced until the stop member 78 engages the wall 80. The stop member 78 can be adjusted anywhere between the maximum and minimum advancement positions in order to control the inward travel of the plunger 72 and thus control the drop volume being produced.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A drop metering device for use in combination with a dropper having a squeezable bottle and a dispenser tip, said device comprising:
a rigid receptacle having an open chamber for removably receiving the bottle along a central axis;
a plunger mounted on the receptacle to reciprocate over a controlled length in a lateral direction relative to the central axis;
whereby inward reciprocation of the plunger deflects a sidewall of the bottle by the controlled length to produce a drop at a controlled speed having a controlled volume.

2. A device as in claim 1, wherein the receptacle is cylindrical and the chamber has a diameter selected to have an interference fit with the bottle of the dropper.

3. A device as in claim 2, wherein the receptacle has a feature which limits the depth to which the bottle can be inserted into the chamber.

4. A device as in claim 3, wherein the feature comprises a bottom.

5. A device as in claim 1, wherein the plunger comprises a lever arm having a remote end which is pivotally attached to the receptacle.

6. A device as in claim 5, further comprising an adjustment mechanism for controlling the length of travel of the plunger in the lateral direction.

7. A device as in claim 6, wherein the adjustment mechanism comprises a rotatable ring disposed between the receptacle and the lever arm, wherein the ring has a varying thickness so rotation of the ring provides a variable limit on the length of travel of the plunger.

8. A device as in claim 7, wherein the ring has a continuously varying thickness from a maximum which prevents the plunger from deflecting the bottle and a minimum which allows a maximum deflection of the bottle.

9. A device as in claim 7, wherein the ring comprises a plurality of posts having different lengths so that individual posts allow the lever to deflect by different amounts.

10. A device as in claim 1, wherein the plunger is slidably received in a laterally oriented slide bearing disposed on a side of the receptacle.

11. A device as in claim 10, further comprising an adjustment mechanism for controlling the length of travel of the plunger in the slide bearing.

12. A device as in claim 11, wherein the adjustment mechanism comprises a stop threadably received on the plunger, wherein the stop can be rotated to axially travel on the plunger to vary the available length of travel.

13. A method for dispensing a drop from a dropper having a squeezable bottle and a dispensing tip, said method comprising:
inserting the bottle of the dropper into a rigid receptacle;
depressing a plunger laterally inwardly by a controlled length relative to the receptacle and toward the bottle, wherein a sidewall of the bottle is deflected by said controlled length to produce a single drop having a controlled volume.

14. A method as in claim 13, further comprising adjusting the controlled length of travel of the plunger.

15. A method as in claim 13, wherein the controlled length is in the range from 0.5 mm to 7.5 mm to produce a drop having a volume in the range from 20 μl to 60 μl.
